(19) United States
(12) Patent Application Publication
(10) Pub. No.: US 2005/0244486 A1
(43) Pub. Date: Nov. 3, 2005

(54) Topical methadone compositions and methods for using the same

(76) Inventors: Larry J. Caldwell, San Jose, CA (US); Bradley S. Galer, West Chester, PA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/115,968

(22) Filed: Apr. 26, 2005

Related U.S. Application Data
(60) Provisional application No. 60/566,686, filed on Apr. 29, 2004.

(51) Int. Cl. A61K 31/137; A61K 9/70

(52) U.S. Cl. 424/449; 514/649

(57) ABSTRACT

Methods and compositions are provided for administering methadone to a subject. Aspects of the invention include employing a topical methadone formulation, e.g., a patch or analogous topical administration formulation. The subject methods and compositions find use in a variety of applications, e.g., in the treatment of a variety of different types of pain.

### CROSS-REFERENCE TO RELATED APPLICATIONS

Pursuant to 35 U.S.C. § 119 (e), this application claims priority to the filing date of the U.S. Provisional Patent Application Ser. No. 60/566,686 filed Apr. 29, 2004; the disclosure of which is herein incorporated by reference.

### BACKGROUND OF THE INVENTION

Pain is a problem that affects a large segment of the population. Pain, such as neuropathic pain and the like, occurs both intermittently and chronically, and can arise in response to variety of stimulants, including stress, injury, toxins in the environment and the like. Whether chronic or acute, pain can take a physical, financial, and emotional toll on those who have pain, as well as on their family and friends.

A variety of therapeutic agents have been developed for use in the treatment of patients suffering from pain, however a safe treatment protocol to effectively treat pain remains elusive. Some agents, such as aspirin, acetaminophen, vasoconstrictors and NSAIDs, e.g. ibuprofen and naprosyn, are administered systemically via conventional routes such as orally and intravenously. Despite the prevalence of this form of treatment for a variety of pain, in some cases, systemic administration by these conventional routes may not be recommended. For example, oral administration of aspirin may result in stomach upset and patient discomfort. Furthermore, the agent may exert subject systemic toxicity which may outweigh any therapeutic benefits provided by the agent. Finally, not all pain is effectively manageable using conventional agents such as aspirin that are formulated for systemic delivery routes such as oral and intravenous routes of administration.

Thus, there is continued interest in the identification of topical formulations that are suitable for use in the treatment of pain. Of interest is the administration of formulations that effectively treat the pain and provide for rapid penetration of an effective amount of the active agent through the skin surface, e.g., to provide for rapid pain relief.

References of Interest

United States patents of interest include: U.S. Pat. No. 6,787,149; 6,720,001; 6,716,449; 6,713,470; 6,638,981;6,586,478; 6,576,650; 6,562,363; 6,538,008; 6,383,471; 6,143,278; 5,989,585; 5,948,389; 5,935,975; 5,883,115; 5,703,101; 5,589,480; 5,580,876; 5,486,362; 5,260,066 and 4,822,617. Also of interest are: Fullerton et al., Acta. Pharm. Nord. (1991) 3:181-182; Gagnon et al., Pain Res. Manag. (2003); Ghosh & Bagherian, Pharm. Dev. & Tech. (1996) 285-291; 8(3):149-54; Hewitt DJ, Clin J Pain. (2000)16(2 Suppl):S73-9; and Morley et al., Palliat. Med. (2003);17(7):576-87.

### SUMMARY OF THE INVENTION

Methods and compositions are provided for administering methadone to a subject. Aspects of the invention include employing a topical methadone formulation, e.g., a patch or analogous topical administration formulation. The subject methods and compositions find use in a variety of applications, e.g., in the treatment of a variety of different types of pain.

### BRIEF DESCRIPTIONS OF THE DRAWINGS

The figures shown herein are not necessarily drawn to scale, with some components and features being exaggerated for clarity.

FIGS. 1A-1E show the results of the stability of methadone in various solvents over a period of time, wherein FIG. 1A reports the time0 results, FIG. 1B report the results at one month, FIG. 1C reports the results at two months, FIG. 1D reports the results at three months and FIG. 1E reports the results at six months.

FIGS. 2A-2C provide graphical results of various in vivo studies, as reviewed in the Experimental Section, below.

### DETAILED DESCRIPTION OF THE INVENTION

Methods and compositions for administering methadone to a subject are provided. Aspects of the subject methods include topically applying a topical methadone formulation that includes an effective amount of methadone, wherein the methadone is the only active agent present in the formulation. Aspects also include contacting a skin surface of a subject with a thermoplastic elastomeric matrix that includes an effective amount of methadone and maintaining the matrix on the skin surface for a period of time sufficient for the methadone to be delivered to the subject. In certain embodiments, the matrix may be a styrene-butadiene-styrene block copolymer matrix or a styrene-isoprene-styrene block copolymer matrix. Also provided are topical formulations that include an effective amount of methadone as the only active agent and thermoplastic elastomeric matrices that include methadone. The subject methods and compositions find use in a variety of different applications, e.g., the treatment of a variety of different types of pain.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Methods recited herein may be carried out in any order of the recited events which is logically possible, as well as the recited order of events.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

As summarized above, the subject invention provides methods and compositions for administering methadone to a subject. In further describing aspects of the invention, aspects of the subject methods are reviewed first in greater detail, followed by a review of other aspects of the invention, e.g., pharmaceutical compositions, kits and systems of the invention, as well as representative applications in which the methods and compositions find use.

Methods

As reviewed above, aspects of the invention provide methods of administering a methadone agent to a subject. By “a methadone agent” is meant methadone (6-dimethylamino-4,4-diphenyl-3-hepatone) and analogs or derivatives thereof. For example, methadone derivatives that are methadone agents include those described in U.S. Pat. No. 5,710,256, the disclosure of which is herein incorporated by reference. In certain embodiments, the methadone agent may be normethadone (6-dimethylamino-4,4-diphenyl-3-hexanone), or a methadone intermediate such as 4-cyano-2-dimethylamino-4,4-diphenylbutane, where it is to be understood that all are contemplated by the subject invention and are included in reference to methadone unless otherwise indicated.

By administering is meant delivering a quantity of methadone to a subject, where features of the invention include systemically administering a quantity of methadone to a subject. By systemically administering is meant that the agent is not acting only locally where the patch is administered, i.e. in tissues underlying the skin region below the patch, but instead is the active medication (methadone) is delivered in a manner that reaches multiple disparate parts of the subject via the circulation. Generally, “subjects” of the invention are “mammals” or “mammalian,” where these terms are used broadly to describe organisms which are within the class mammalia. Of interest is the treatment of primates with the subject methods, (e.g., humans, chimpanzees, and monkeys), where the subject methods are particularly suited for use in the treatment of humans suffering from neuropathic pain, nociceptive pain, inflammatory pain, acute pain, chronic pain, cancer pain, and other types of pain, as described above.

A feature of embodiments of the invention is that the agent is systemically administered to the subject in a manner that provides for a therapeutic level of agent in the blood of the subject for an extended period of time. While the “therapeutic level” achieved in a given embodiment may vary, in certain representative embodiments the therapeutic analgesia level of agent that is achieved by practice of the subject methods is a blood level of agent that ranges from about 50 ng/ml to about 500 ng/ml,. The therapeutic level for analgesia that is achieved following a given administration may last for an extended period of time, e.g., from about 4 hrs to about 24 hrs. In certain embodiments, administration according to the subject methods results in a blood level profile of the agent characterized by at least a first phase characterized by an initial increase in blood level over an initial period of time ranging from about 0.1 to about 10 hours, such as from about 0.25 to about 8 hours, followed by second phase characterized by a gradual decrease in level over an extended period of time, e.g., ranging from about 2 hours to about 24 hours or longer, where the second phase is longer than the first phase by at least about 2-fold, such as by at least about 5-fold or even 10-fold or more.

A feature of embodiments of the invention is that the agent is administered as described above using a topical formulation. As reviewed in greater detail below, embodiments of the invention provide for the above administration features from a topical formulation having a dosage or amount of a methadone agent that ranges from about 0.10% to about 30.0% (w/w) of methadone agent, e.g., from about 0.5% to about 15.0% (w/w), e.g., from about 1.0% to about 5.0% (w/w).

In the broadest sense, any convenient topical methadone formulation that provides for the requisite penetration of the methadone in the formulation through the skin surface to the target area of the subject may be employed. The topical formulation may be a gel, lotion, spray, ointment, cream, patch, tape, plaster and the like. In certain embodiments, the methadone is present in a matrix, where matrices of interest include, but are not limited to, macromolecular matrices such as thermoplastic elastomeric matrices, e.g., styrene-butadiene-styrene block copolymer matrices, styrene-isoprene-styrene block copolymer matrices, and the like.

In certain embodiments, the topical methadone formulations are those in which the sole active agent is a methadone active agent. As such, these embodiments are characterized in that no other active agents are present in the topical formulations. The topical methadone formulations employed in the subject methods are formulations that includes an effective amount of a methadone agent, where in many embodiments the agent is the only active agent in the topical formulation. Accordingly, as used herein, a “topical methadone formulation” and analogous terms is meant a formulation that includes a methadone agent and is capable of administration of the methadone agent to a subject through the surface of the subject's body part.

While the amount of active agent in the subject formulations vary, in many embodiments the amount of methadone agent present in the topical formulations is an amount that is effective for treating a subject for pain, including an amount effective for at least reducing the frequency and/or intensity of the pain, i.e., is present in at least a pain-reducing amount in the formulations. In certain embodiments, methadone may be present in a pain preventing amount such that the magnitude or intensity of pain is not only reduced, but the pain is all together eliminated, at least for a period of time, by the amount of active agent present in the formulation. In certain embodiments, the amount of methadone present may be sufficient to solely act locally, solely act systemically or may be in an amount that is sufficient to act both locally and systemically to treat pain. As reviewed above, in many embodiments administration is systemic. In this manner, methadone may be in an amount sufficient to act as a μ-opioid agonist and/or an N-methyl-D-asparatate (“NMDA”) receptor antagonist, locally, systemically or both locally and systemically. Embodiments may include from about 0.10% to about 30.0% (w/w) of methadone agent, e.g., from about 0.5% to about 15.0% (w/w), e.g., from about 1.0% to about 5.0% (w/w).

As noted above, the topical methadone formulations may be in any suitable form that enables the methadone agent to be effectively delivered topically. By “topical”, “topical administration” and analogous terms is used herein in its conventional meaning to refer to direct contact with the surface of the body, such as to the skin, eyes, mucosa and lips, which may be in or on any part of the body, including but not limited to the epidermis, any other dermis, or any other body tissue. Topical administration or application means the direct contact of the methadone formulation with tissue, such as skin or membrane such as the cornea, or oral, vaginal or buccal mucosa. Topical administration also includes application to hardened tissue such as teeth and appendages of the skin such as nails and hair. In many embodiments, the topical formulations are those that are formulated to be applied to an intact, keratinized skin surface of a subject.

The methadone agent may be formulated into topical preparations in solid, semi-solid, liquid or gaseous forms, such as, but not limited to, gels, lotions, emulsions, creams, pastes, jellies, paints, powders, plasters, ointments, sprays such as aerosols, or may be in the form of a “finite” carrier, i.e., a non-spreading substance that retains its form, such as a patch, bioadhesive, dressing and bandage, e.g., present on a surface of a support. The subject topical formulations may be aqueous or non-aqueous and may be formulated as a solution, emulsion or a suspension.

Topical formulations may include one or more of a penetrating agent, thickener, diluent, emulsifier, dispersing aid, or binder. For example, a topical methadone formulation may be formulated with or for use with a penetration enhancer. Penetration enhancers, which include chemical penetration enhancers and physical penetration enhancers, facilitate delivery of the compound through the skin, and may also be referred to as “permeation enhancers” interchangeably. Physical penetration enhancers include, for example, electrophoretic techniques such as iontophoresis, use of ultrasound (or “phonophoresis”), and the like. Chemical penetration enhancers are agents administered either prior to, with, or immediately following administration of the active agent, which increase the permeability of the skin, particularly the stratum corneum, to provide for enhanced penetration of the active agent through the skin.

Compounds that may be used to enhance skin permeability include, but are not limited to, the sulfoxides dimethylsulfoxide (DMSO) and decylmethylsulfoxide (C₁₀ MSO); ethers such as diethylene glycol monoethyl ether, dekaoxyethylene-oleylether, and diethylene glycol monomethyl ether; surfactants such as sodium laurate, sodium lauryl sulfate, cetyltrimethylammonium bromide, benzalkonium chloride, Poloxamer (231, 182, 184), Tween (20, 40, 60, 80) and lecithin; the 1-substituted azacycloheptan-2-ones, particularly 1-n-dodecylcyclazacycloheptan-2-one; alcohols such as ethanol, propanol, octanol, benzyl alcohol, and the like; petrolatums, such as petroleum jelly (petrolatum), mineral oil (liquid petrolatum), and the like; fatty acids such as C₈-C₂₂ and other fatty acids (e.g., isostearic acid, octanoic acid, oleic acid, lauric acid, valeric acid); C₈-C₂₂ fatty alcohols (e.g., oleyl alcohol, lauryl alcohol); lower alkyl esters of C₈-C₂₂ fatty acids and other fatty acids (e.g., ethyl oleate, isopropyl myristate, butyl stearate, methyl laurate, isopropyl myristate, isopropyl palmitate, methylpropionate, ethyl oleate); monoglycerides of C₈-C₂₂ fatty acids (e.g., glyceryl monolaurate); tetrahydrofurfuryl alcohol polyethylene glycol ether; 2-(2-ethoxyethoxy)ethanol; diethylene glycol monomethyl ether; alkylaryl ethers of polyethylene oxide; polyethylene oxide monomethyl ethers; polyethylene oxide dimethyl ethers; di-lower alkyl esters of C₆-C₈ diacids (e.g., diisopropyl adipate); ethyl acetate; acetoacetic ester; polyols and esters thereof such as propylene glycol, ethylene glycol, glycerol, butanediol, polyethylene glycol, and polyethylene glycol monolaurate; amides and other nitrogenous compounds such as urea, dimethylacetamide (DMA), dimethylformamide (DMF), 2-pyrrolidone, N-alkylpyrrolidone, e.g., 1-methyl-2-pyrrolidone; ethanol amine, diethanol amine and triethanolamine; terpenes; alkanones, and organic acids, particularly salicylic acid and salicylates, citric acid and succinic acid. Additional chemical and physical penetration enhancers are described in, for example, Transdermal Delivery of Drugs, A. F. Kydonieus (ED) 1987 CRL Press; Percutaneous Penetration Enhancers, eds. Smith et al. (CRC Press, 1995); Lenneruas et al., J Pharm Pharmacol 2002;54(4):499-508; Karande et al., Pharm Res 2002;19(5):655-60; Vaddi et al., J Pharm Sci 2002 July;91(7):1639-51; Ventura et al., J Drug Target 2001;9(5):379-93; Shokri et al., Int J Pharm 2001;228(1-2):99-107; Suzuki et al., Biol Pharm Bull 2001;24(6):698-700; Alberti et al., J Control Release 2001;71(3):319-27; Goldstein et al., Urology 2001;57(2):301-5; Kiijavainen et al., Eur J Pharm Sci 2000;10(2):97-102; and Tenjarla et al., Int J Pharm 1999;192(2):147-58.

Where a chemical penetration enhancer is employed, the penetration enhancer is selected for compatibility with the methadone, and is present in an amount sufficient to facilitate delivery of the c methadone through skin of a subject.

Topical application of the subject methadone formulations may be accomplished by a variety of methods, including, but not limited to, rubbing, spraying, or the like, a formulation of the invention onto an area of intact skin, positioning a matrix (such as a macromolecular matrix, e.g., a thermoplastic elastomer matrix, and the like) that includes an amount of methadone onto an area of intact skin, and the like. Methadone formulations suitable for transdermal administration may also be delivered by iontophoresis or the like.

As noted above, embodiments include topical methadone formulations formulated as applicator sticks, solutions, suspensions, emulsions, gels, lotions, creams, ointments, pastes, jellies, paints, powders, sprays such as aerosols, emulsions, plasters, etc. In certain embodiments, the methadone formulations may be in the form of an adhesive base such as a pressure sensitive adhesive base, e.g., a thermoplastic elastomeric matrix and the like, e.g., as a discrete patch, bioadhesive or film or plaster or the like, adapted to remain in intimate contact with a surface of a body part such as the epidermis of a subject for a period of time. For example, such matrices may include a base or matrix component, e.g., macromolecular matrix such as a thermoplastic elastomer component, in which an effective amount of methadone is retained. The base or matrix layer may be operatively associated with a support or backing. Embodiments include macromolecular matrices that include an effective amount of methadone. The macromolecular substance that may be employed in accordance with the subject invention may be either a natural macromolecular substance or a synthetic macromolecular substance. It may be adhesive or nonadhesive e.g., it may be inherently adhesive or inherently non-adhesive. If a nonadhesive substance is employed, an adhesive component may be added to it to impart adhesive properties to attain a suitable level of adhesiveness. Examples of macromolecular substances that may be employed in the subject invention include, but are not limited to, natural rubber, polyisoprene, polybutadiene, styrene-isoprene-styrene (“SIS”) block copolymers, styrene-butadience-styrene (“SBS”) block copolymers, polyacrylic esters, polymethacrylic esters, acrylic ester methacrylic ester copolymers, acrylic acid acrylic ester-vinyl acetate copolymers, petroleum resins, and the like. These macromolecular substances may be used alone or in combination of two or more, wherein many embodiments two or more macromolecular substances may be employed as a matrix. The matrices are typically easily released from the skin without appreciable pain or irritation. While embodiments of the subject methadone-containing matrices compositions are inherently adhesive to a skin surface, they are advantageously sufficiently cohesive so as to be easily removable from the skin surface.

In many embodiments a thermoplastic elastomer is a main component of a matrix. The thermoplastic elastomer is generally a block copolymer that may be represented by the following general formula:
A-B-A or (A-B-)ₙ^{x}
wherein “A” is substantially a monovinyl-substituted aromatic compound polymer block,

“B” is a substantially conjugated diolefin polymer block,

“n” is an integer of from about 3 to about 7, and

“X” indicates a residue derived from a polyfunctional compound with which 3-7 (n) polymer chains (A-B) are combined.

The block copolymer of the formula may represent a TR block copolymer, a radial TR block copolymer or a mixture thereof in certain embodiments.

The above monovinyl-substituted aromatic compounds include, but are not limited to, styrene, o- or p-vinyltoluene, methylstyrene and ethylstyrene. The conjugated diolefins include, but are not limited to, 1,3-butadiene, 1,3-pentadiene and isoprene. A combination of styrene with 1,3-butadiene, and a combination of styrene with isoprene are combinations that may be employed. Embodiments include matrices of block A which may be a polymer of styrene and of block B which is a polymer of isoprene or butadiene.

The end block A in the above block copolymer may be contained therein in an amount ranging from about 10% to about 80% by weight of the block copolymer, e.g., from about 14% to about 22%.

Other, optional components of a matrix (or other topical methadone formulations) may include, but are not limited to, solvents, resins, waxes, e.g., liquid paraffin and the like, anti-oxidants, e.g., dibutylhydroxytoluene and the like, etc, as well as other optional components described herein. For example, exemplary solvents that may be employed include, but are not limited to, mineral oil, N-methyl-2-pyrrolidone, diisopropyl adipate, DEET, PEG, Di (propylene glycol), dehydrated alcohol, water, and the like. In certain embodiments, the formulations include DEET, where DEET is present in an amount ranging from about 1 to about 30%, including about 5 to about 25%, including about 5 to about 20%, such as from about 7.5 to about 15%, e.g., 10%. Exemplary resins that may be employed include, but are not limited to, alicyclic saturated hydrocarbon resin, hydroxyl terminated polybutadiene, and the like.

In certain embodiments, a topical methadone formulation used in the subject invention may be prepared from water-insoluble components, or salts thereof, such as aqueous base emulsions. In such embodiments, the formulations may contain a sufficient amount of pharmaceutically acceptable emulsifying agent to emulsify the component(s). Useful emulsifying agents include, but are not limited to, phosphatidyl cholines, lecithin, and the like. Other additives, such as pH-adjusting additives may also be included in the methadone formulations. For example, pH-adjusting agents include, but are not limited to, acids, such as hydrochloric acid, bases or buffers, such as sodium lactate, sodium acetate, sodium phosphate, sodium citrate, sodium borate, or sodium gluconate. Furthermore, microbial preservatives may be used. Microbial preservatives that may be employed include, but are not limited to, methylparaben, propylparaben, and benzyl alcohol.

In certain embodiments, a given matrix may be present on a backing layer or support. The support is generally made of a flexible material which is capable of fitting in the movement of the human body and includes, for example, various non-woven fabrics, woven fabrics, spandex, flannel, or a laminate of these materials with polyethylene film, polyethylene glycol terephthalate film, polyvinyl chloride film, ethylene-vinyl acetate copolymer film, polyurethane film, and the like. By “flexible” it is meant that the support may be substantially bent or folded without breaking, tearing, ripping, etc.

The support may be porous or non-porous, but is typically non-porous. In certain embodiments, the backing layer is substantially impermeable to the matrix composition, methadone and fluids, e.g., any fluids exuded from the application site. Such impermeability of the backing increases the effectiveness an efficiency of the methadone-containing matrix composition. For example, substantial impermeability to methadone serves to enhance or increase the penetration of the methadone into the skin.

The length and width dimensions of the support are typically substantially commensurate, including exactly commensurate, with the length and width dimensions of the methadone-containing matrix composition with which it is associated. The support layer may have a thickness ranging from about 10 μm to about 1000 μm, but may be less than 10 μm and/or greater than 1000 μm in certain embodiments.

In addition to the methadone-containing matrix composition and the support layer (if present), a release film may be positioned on the surface of the methadone-containing matrix composition opposite the backing which provides for protection of the methadone-containing matrix composition from the environment. The release film may be any convenient material, where representative release films include polyesters, such as PET or PP, and the like. A methadone-containing matrix/backing layer transdermal drug delivery system may be fabricated using any convenient protocol. One convenient protocol for fabrication of the subject methadone-containing matrix/backing layer transdermal drug delivery system includes preparing a paste-like mixture through the uniform mixing of the aforementioned ingredients and then coating the paste onto a support, followed by cutting of the resultant product to the specified size to obtain the desired methadone-containing matrix/backing layer transdermal drug delivery system. The amount of methadone-containing matrix layer present on a backing layer may vary, where in certain embodiments the amount may range from about 500 grams/meters² to about 10,000 grams/meters².

The shape of the methadone-containing matrix/backing layer transdermal drug delivery system may vary, where representative shapes include, but are not limited to, square, rectangle, oval, circle, triangular, etc. The size of a methadone containing matrix/backing layer transdermal drug delivery system may also vary, where in many embodiments the size may range from about 1 to about 1000 cm² or more, e.g., in certain embodiments ranges from about 10 to about 300 cm², e.g., from about 20 to about 200 cm², e.g., about 130 cm² to about 150 cm². In certain embodiments, the surface area is sufficient to cover the entire truck of a subject. Accordingly, the surface area may range from about 1000 cm² to about 5000 cm² or more, where in certain embodiments the dimensions of a subject methadone-containing matrix/backing layer transdermal drug delivery system may be about 1 m by about 1 m. For a more detailed description of the fabrication protocol, see for example U.S. Pat. No. 5,827,529, the disclosure of which is herein incorporated by reference.

An exemplary method of fabricating a methadone-containing matrix /backing layer transdermal drug delivery system includes incorporating methadone in a macromolecular matrix such as an SIS block copolymer matrix or the like, and spreading the resultant preparation upon a surface of a backing layer to provide a methadone-containing matrix layer on the backing layer. A releasable liner or cover may be applied to the methadone-containing matrix present on the backing layer. The resultant transdermal system may be cut to smaller sizes and/or various shapes if desired.

For example, a mixture containing one or a combination of thermoplastic elastomers and one or more other components such as those described herein, may be heated to soften or melt the mixture. The resultant mixture may then be cooled and a suitable amount of methadone may be added to the mixture and mixed for a period of time to disperse the methadone throughout the mixture. The thus obtained methadone-containing matrix may then be spread or coated on a support by the use of a doctor roll, reverse roll coater, slot die coater, knife coater, and the like. It should be noted that the above manufacturing protocols are merely representative. Any convenient protocol that is capable of producing a methadone-containing matrix/backing layer transdermal drug delivery system of the subject invention may be employed.

Regardless of the form of the topical methadone formulation, in practicing the subject methods, a subject topical methadone formulation is applied to the surface of a subject's body, e.g., a skin surface of the subject, in a manner sufficient to provide for penetration of an affective amount of methadone through the skin. The topical methadone formulation may be applied directly over the source of pain or directly to the skin surface associated with the pain, or may not be directly applied over the source of the pain. In those embodiments where a methadone-containing matrix /backing layer transdermal drug delivery system is employed, the system is first removed from any packaging that may be present, and then the protective layer, if present, is removed, thereby exposing the methadone-containing matrix. The methadone-containing matrix/backing layer transdermal drug delivery system is then positioned on a surface of the body such as a skin surface of a subject. As mentioned above, in certain embodiments the methadone-containing matrix delivery systems are self-adhesive, i.e., inherently adhesive, and thus may be fixed in a position, i.e., removably bonded to the skin surface, without the use of additional adhesives or other means to hold the methadone-containing matrix in place over the formulation. As noted above, in certain embodiments the topical formulation may be in the form of a cream or the like and such is dispensed from the dispenser packaging from which it is provided and spread onto a surface of the body such as a skin surface and then optionally occluded with a matrix (which may or may not be present on a support) that does not include methadone in the matrix, such as a macromolecular matrix as described above that is absent any methadone (i.e., a “blank matrix”). That is, in certain embodiments, a matrix layer that does or does not include methadone may be positioned over a skin surface that has a topically applied methadone formulation immediately applied thereto.

A topical methadone formulation of the subject invention may be employed to act locally (peripherally), systemically, or locally and systemically, where such is determined at least in part on the particular methadone formulation employed and the like. Accordingly, in certain embodiments the subject methods include topically applying an effective amount of methadone to a skin surface directly over the source of pain, to treat a subject for pain by solely acting locally as a local mu-opioid agonist and a local NMDA receptor antagonist. In certain embodiments, the subject methods include topically applying an effective amount of methadone to a skin surface, but not directly over the source of the pain, to treat the subject for pain by solely acting systemically (such as to result in low systemic activity) as a systemic mu-opioid agonist and a systemic NMDA receptor antagonist. In certain other embodiments, the subject methods include topically applying an effective amount of a methadone agent to a skin surface directly over the source of pain, to treat a subject for pain by acting locally as a local mu-opioid agonist, locally as an NMDA receptor antagonist, systemically as a local mu-opioid agonist, and systemically as an NMDA receptor antagonist.

The topical methadone formulation may be applied to any convenient topical site. Topical sites of interest include, but are not limited to: arms, legs, joints, face, neck, torso, etc. The topical formulation can be applied to one or more distinct regions, depending on the pain origin.

The amount of surface area upon which a methadone formulation is applied may vary depending on the particular pain condition being treated, the application site, etc. The surface area that is covered by the topical formulation must be sufficient to provide for the effective and efficient administration of a desired amount of methadone and in many embodiments may range from about 1 to about 1000 cm² or more, e.g., in certain embodiments ranges from about 10 to about 300 cm², e.g., from about 20 to about 200 cm², e.g., about 130 cm² to about 150 cm². In certain embodiments, the surface area is sufficient to cover the entire truck of a subject. Accordingly, the surface area may range from about 1000 cm² to about 5000 cm² or more, where in certain embodiments the surface upon which the topical formulation is applied may be about 1 m by about 1 m. In practicing the subject methods, a topical formulation may be applied a single time or a plurality of times over a given time period, where the dosing schedule may be daily, weekly, biweekly, monthly, etc. For example, certain topical formulations may be applied two or more times a day, two or more tines a week, etc.

The amount of methadone agent applied is sufficient to provide for the desired reduction in at least one aspect of pain, e.g., frequency and/or intensity of the pain. The exact amount of topical methadone that is applied may be determined empirically. For solutions, dispersions, gels, lotions, creams and the like, the methadone formulation will be spread over the region and a covering optionally applied thereto, as noted above. For methadone formulations in the form of a transdermal system that includes a matrix such as an SIS and/or SBS block copolymer matrix present on a support, an appropriate sized system will be placed over the region comprising the application site such as a skin site. The formulation is maintained in place for a period of time sufficient for the desired amelioration in symptoms, e.g., reduction in pain, to occur. The particular period of time during which the topical formulation is maintained at the application site depends on a variety of factors such as, but not limited to, the nature of the pain, the subject, e.g., the sensitivity of the subject to the methadone, etc., but generally the formulation is maintained in place for at least about 30 min, e.g., at least about 1 hr, e.g., at least about 4 hr, where the formulation may be maintained in place for as long as about 8 hr to about 12 hr or longer and in certain embodiments the period of time may range from about a few hours to about a few days or more, e.g., about one or more days, e.g., about a week or more. These time periods may represent total treatment time periods, i.e., the total amount of time an area of skin is treated according to the subject methods, or may be a period of time of a first treatment and/or any successive treatments at a particular application site, such that additional treatments according to the subject methods may be performed following a first treatment at a particular application site, e.g., immediately thereafter or after a period of time has passed. Successive treatments may include application of an effective amount of the same topical methadone formulation used in a previous application, an effective amount of a different dosage of methadone used in a previous application, an effective amount of the same methadone formulation present in a different topical formulation form (e.g., a cream instead of a transdermal matrix system), etc.

Accordingly, in certain embodiments immediately or after a sufficient period of time has elapsed, the subject methods may be repeated one or more times so that an additional topical methadone formulation (which may be the same or a different methadone formulation than used previously) may be applied to an application site. For example, where methadone-containing matrix/backing layer transdermal drug delivery systems are employed, the amount of time a methadone-containing matrix/backing layer transdermal drug delivery system may be replaced with another, for example during the course of a day, may range from about 1 time to about two times, and in certain embodiments a methadone-containing matrix/backing layer transdermal drug delivery system may be replaced more than two times in a day. For example, in certain embodiments, a system may be changed about once every 24 hours or so. In certain embodiments a system may be worn only during the waking hours and is removed during sleep. Embodiments include gradually decreasing the strength of methadone employed over time by applying decreasing amounts of methadone to the subject. For example, using a first system of a first amount or dosage of methadone for a period of time (or a plurality of such systems), e.g., every day for about 1 to about 4 weeks, and then a second, or weaker strength methadone system (or a plurality of such systems) thereafter, which may be followed by a third, or weaker strength methadone system (or a plurality of such systems) thereafter, etc., wherein the effective amount of methadone delivered to a subject is gradually decreased over time by using methadone formulations of varying amounts of methadone.

In those embodiments where a methadone-containing matrix/backing layer transdermal drug delivery system is used, the methadone-containing matrix/backing layer transdermal drug delivery system is removed from the application site once a sufficient amount of time has elapsed (and replaced by another or other methadone formulation if desired). The nature of the methadone-containing matrix/backing layer transdermal drug delivery system enables it to be easily and non-traumatically removed from the application site by simply peeling the methadone-containing matrix/backing layer transdermal drug delivery system away from the site. Upon removal, the methadone-containing matrix/backing layer transdermal drug delivery system is removed intact, i.e., the methadone-containing matrix/backing layer transdermal drug delivery system does not leave debris at the site.

A feature of the subject methods is that upon application of the topical methadone composition, the methadone agent present therein penetrates the surface of the body such as the surface of skin to achieve the desired result, e.g., treat the subject for pain.

Embodiments of the subject invention include one or more additional steps, e.g., diagnosing the subject as a subject in need of administration of a methadone agent.

In certain embodiments, the topical formulation that is employed is one that has been stored for an extended period of time, e.g., at least about 1 month or longer, at least about 2 months or longer, at least about 3 months or longer, at least about 4 months or longer, at least about 5 months or longer, at least about 6 months or longer, at least about 9 months or longer, at least about 12 months or longer, at least about 24 months or longer, etc, under standard storage conditions, e.g., as reported in the Experimental Section below, or conditions considered in the art as analogous thereto. For representative conditions, see also the Experimental Section, below (e.g., about at temperature 25C+/−2C). A given formulation is considered storage stable if the amount of active agent following the storage time period is at least about 85%, such as at least about 90%, including at least about 95%, e.g., as determined using the HPLC protocol described in the Experimental Section, below.

Utility

Applications of the subject methods include topically administering an effective amount, i.e., a therapeutically effective amount, of a methadone to a subject. By “effective amount” and analogous terms is meant a dosage sufficient to achieve the desired result, e.g., treat the subject for pain for a period of time. The effective amount will vary with the age and physical condition of the subject, type and severity of the pain being treated, the duration of the treatment, the nature of any concurrent treatment, the form of the methadone formulation, the pharmaceutically acceptable carrier used if any, and analogous factors within the knowledge and expertise of those skilled in the art. Such dosages may be determined in accordance with routine pharmacological procedures known to those skilled in the art. The frequency of administration of the subject topical methadone formulations may range from about 1 time per day to multiple times per day, e.g., about 2 times or more per day or as necessary to treat or otherwise prevent, control or manage pain, e.g., at least reduce the frequency and/or intensity of the pain for a period of time. The duration of therapy depends on the type of pain being treated, etc., and may range from as short as about 24 hours (or less in certain embodiments) to as long as the life of the subject.

The above-described invention finds use in a variety of different applications, including but not limited to: treatment of a variety of different conditions, e.g., drug addiction, pain, etc., where in many representative embodiments the subject methods are employed for treating a subject suffering from pain. The subject methods may be employed to treat a variety of different types of pain, including, but not limited to, neuropathic pain, nociceptive pain, inflammatory pain, acute pain, chronic pain, cancer pain, and other types of pain. The subject methods may also be used as a safe and efficacious treatment for narcotic withdrawal and dependence, e.g., to treat a subject for opioid addiction such as heroine addiction and the like.

A variety of subjects are treatable according to the subject methods. Generally such subjects are “mammals” or “mammalian,” where these terms are used broadly to describe organisms which are within the class mammalia. Of interest is the treatment of primates with the subject methods, (e.g., humans, chimpanzees, and monkeys), where the subject methods are particularly suited for use in the treatment of humans suffering from neuropathic pain, nociceptive pain, inflammatory pain, acute pain, chronic pain, cancer pain, and other types of pain, as described above.

As noted above, the subject invention finds use in the treatment of pain. By “treatment” and analogous terms it is meant at least an amelioration of the pain for a period of time, where amelioration is used in a broad sense to refer to at least a reduction in the magnitude or intensity and/or frequency of the pain as evaluated according to the pain assessment tool commonly known as the Pain Relief Score protocol (where 0-worse pain; 1-no change; 2-slight improvement; 3-moderate improvement; 4-alot of improvement; 5-complete relief). In many embodiments, the magnitude of reduction in pain intensity may be at least about 10% (slight relief), e.g., at least about 25% (slight-moderate relief), e.g., at least about 50% (moderate relief), where magnitude of reduction may be as high as about 75%, about 80%, about 95% or higher (a lot of relief), including a complete cessation of pain (complete relief). The period of time may vary, where in certain embodiments the period of time may range from about 1 hour to about 24 hours of longer, e.g., a period of time may be about 3 hrs, e.g., at least about 6 hrs in certain embodiments, e.g., at least about 12 hrs or longer, e.g., about 16 hrs, about 24 hrs, or longer. Treatment as used herein also includes situations where the pain is completely inhibited, e.g., prevented from happening, or stopped, i.e., terminated, such that the subject no longer suffers from the pathological condition, at least for a period of time. As such, application and maintenance of the topical methadone formulation as described above results in at least an amelioration or reduction in the magnitude and/or frequency of pain, including a complete cessation or removal of the pain for a period of time, e.g., for about 1 hour or more, e.g., a period of time that may be about 3 hrs, e.g., at least about 6 hrs in certain embodiments, e.g., at least about 12 hrs or longer, e.g., about 16 hrs, about 24 hrs, or longer.

In many embodiments, the intensity of pain associated is at least reduced, where in certain embodiments the pain may be completely eliminated or inhibited, e.g., prevented from happening, or stopped, e.g., terminated, such that the subject no longer suffers from the pain, at least for a period of time that may be about 3 hrs, e.g., at least about 6 hrs in certain embodiments, e.g., at least about 12 hrs or longer, e.g., about 16 hrs, about 24 hrs, or longer.

Topical Methadone Formulations

Also provided are topical methadone formulations that include an effective amount of a methadone active agent, as described above, where the topical methadone formulations are present in a configuration that is tailored for its use in the treatment of pain according to the subject methods. For example, the topical formulations of methadone may in the form of a gel, lotion, spray, paint, ointment, cream, patch, tape, plaster and the like, as described above. In certain embodiments, the methadone formulations are present as a macromolecular matrix, where matrices of interest include, but are not limited to, thermoplastic elastomer matrices such as styrene-butadiene-styrene block copolymer matrices, styrene-isoprene-styrene block copolymer matrices, and the like, which may be present on a backing layer. In certain embodiments, methadone is the sole active agent present in the topical preparation and in other embodiments more than one active agent (methadone and one or more other active agents) may be present.

Embodiments include methadone-containing matrix/backing layer transdermal drug delivery systems and analogous structures that are shaped specifically with respect to the target epidermal location of their intended application, e.g., to cover the requisite surface area of the target location as described above, e.g., as rectangular, square, round, oval or other shapes configured to cover a target skin surface application site in a manner described above. The amount of active methadone present in the formulation may vary depending on the nature of the formulation, but in many embodiments may range from about 0.1% to about 30.0% (w/w), e.g., from about 0.5% to about 15.0% (w/w), e.g., from about 1.0% to about 5.0% w/w.

Kits

Also provided are kits for practicing the subject methods. The subject kits may vary greatly in regards to the components included. The subject kits at least include a topical methadone formulation for use in practicing the subject methods. The topical methadone formulation may be in any suitable form, e.g., a gel, lotion, spray, ointment, cream, patch, paint, tape, plaster and the like, as described above. In certain embodiments, a kit may include methadone formulations in the form of a macromolecular matrix, such as methadone included in a thermoplastic elastomer matrix, e.g., styrene-butadiene-styrene block copolymer matrix, styrene-isoprene-styrene block copolymer matrix, and the like, which may be present on a backing layer, and described above. In certain embodiments, a kit may include topical methadone formulation and a macromolecular matrix such as a thermoplastic elastomer matrix and the like, e.g., styrene-butadiene-styrene block copolymer matrix, styrene-isoprene-styrene block copolymer matrix, and the like, such that the matrix does not include methadone at all (i.e., the matrix may be a “methadone-blank matrix”).

The amount of topical methadone formulation provided in a kit may be sufficient for a single application or for multiple applications. For example, where the formulation is present as a cream or the like, an amount suitable for multiple applications may be provided, e.g., packaged in a single container, e.g., a single tube, bottle, vial, and the like, or one or individually packaged in separate vials, tubes, and the like. Where the formulation is in the from of methadone-containing matrix/backing layer transdermal drug delivery system, a plurality of methadone-containing matrix/backing layer transdermal drug delivery systems may be provided in a kit, each individually packaged. In such embodiments having more than one methadone-containing matrix/backing layer transdermal drug delivery system, a large number of methadone-containing matrix/backing layer transdermal drug delivery systems may be sealed together within a single packaging. However, typically each methadone-containing matrix/backing layer transdermal drug delivery system present in the kit is sealed in an individual package so that one methadone-containing matrix/backing layer transdermal drug delivery system may be removed from its packaging and used while the packaging of any other methadone-containing matrix/backing layer transdermal drug delivery system of the kit remains intact or un-breached. Where a number of systems (or other form of topical methadone formulation) are provided, the dosage of methadone may vary, e.g., for application of a decreasing amount of methadone to a subject over time.

Some or all components of the subject kits may be packaged in suitable packaging to maintain sterility. In many embodiments of the subject kits, the components of the kit are packaged in a kit containment element to make a single, easily handled unit, where the kit containment element, e.g., box or analogous structure, may or may not be an airtight container, e.g., to further preserve the sterility of some or all of the components of the kit. The subject kits may include instructions for how to use the topical methadone formulations in order to deliver the methadone to a subject to treat pain. The instructions may be recorded on a suitable recording medium or substrate. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

The following examples are offered by way of illustration and not by way of

### Experimental

The following examples are put forth so as to provide those of ordinary skill in the art with a complete disclosure and description of how to make and use the present invention, and are not intended to limit the scope of what the inventors regart as their invention. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

I. Solubility and Stability of Methadone in Different Solvents

In this experiment, the solubility of methadone in a number of different solvents was evaluated. The solvents evaluated were mineral oil, N-methyl-2-pyrrolidone, diisopropyl adipate, DEET, PEG, Di (propylene glycol), dehydrated alcohol and water. The solubility of the solutions was evaluated at 3 hours, 6 hours, 24 hours and 48 hours. The stability of the solutions was also evaluated.

Results

solubility

| | Time | | | |
|---|---|---|---|---|
| | Point | Amount | Dilution | Solubility |
| Solvent | (hour) | (μg/mL) | Factor | (mg/mL) |
| Mineral oil | 3 | 0.369 | 100000 | 39.9 |
| | 6 | 0.372 | 100000 | 37.2 |
| | 24 | 0.202 | 200000 | 40.4 |
| | 48 | 0.202 | 200000 | 40.4 |
| N-methyl-2- | 3 | 5.25 | 50000 | 262 |
| pyrrolidone | 6 | 5.40 | 50000 | 270 |
| | 24 | 6.94 | 50000 | 347 |
| | 48 | 6.84 | 50000 | 342 |
| diisopropyl | 3 | 6.86 | 50000 | 343 |
| adipate | 6 | 6.90 | 50000 | 345 |
| | 24 | 18.0 | 20000 | 360 |
| | 48 | 16.2 | 20000 | 324 |
| DEET (97%) | 3 | 2.48 | 50000 | 124 |
| | 6 | 2.50 | 50000 | 125 |
| | 24 | 6.87 | 20000 | 137 |
| | 48 | 6.60 | 20000 | 132 |
| PEG - 400 | 3 | 0.794 | 50000 | 39.7 |
| | 6 | 0.798 | 50000 | 39.9 |
| | 24 | 0.775 | 50000 | 38.8 |
| | 48 | 0.736 | 50000 | 36.8 |
| Di(propylene | 3 | 5.62 | 10000 | 56.2 |
| glycol) | 6 | 5.65 | 10000 | 56.5 |
| | 24 | 2.75 | 20000 | 54.9 |
| | 48 | 2.93 | 20000 | 58.7 |
| Dehydrated | 3 | 11.4 | 10000 | 114 |
| Alcohol | 6 | 11.3 | 10000 | 113 |
| | 24 | 5.77 | 20000 | 115 |
| | 48 | 5.89 | 20000 | 118 |
| water | 3 | 0.217 | 1000 | 0.217 |
| | 6 | 0.228 | 1000 | 0.228 |
| | 24 | 0.224 | 1000 | 0.224 |
| | 48 | 0.225 | 1000 | 0.225 |

stability

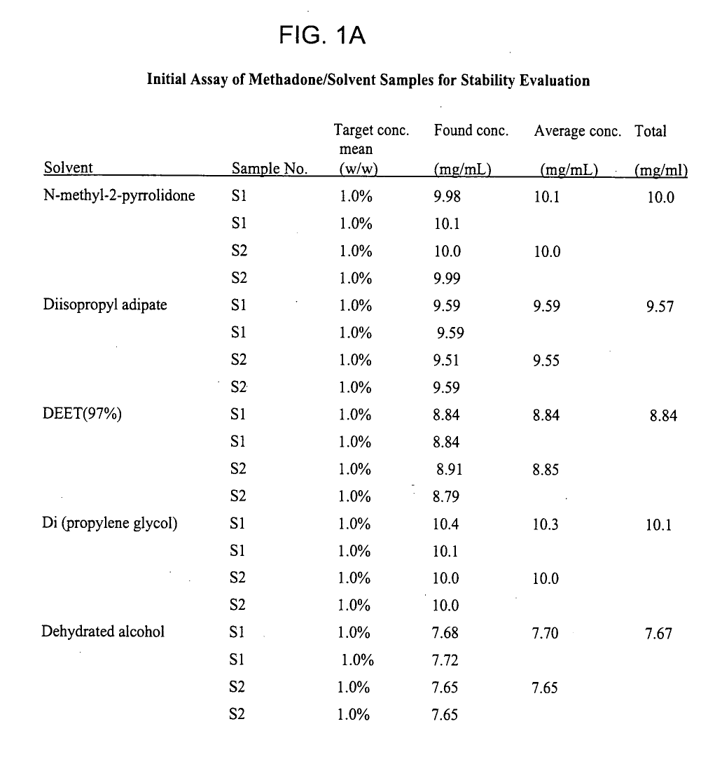

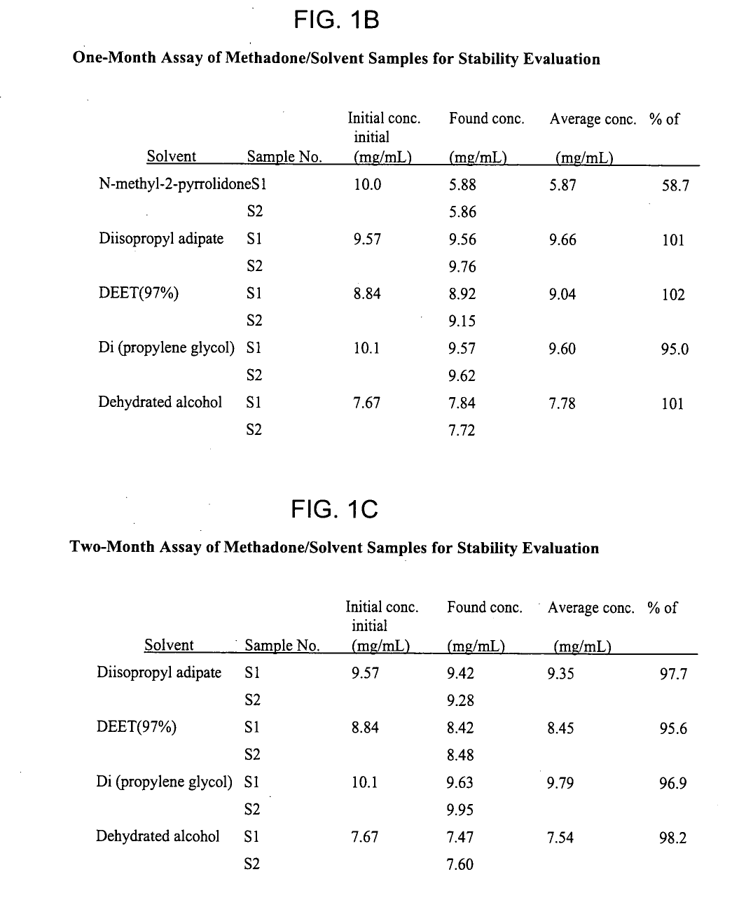

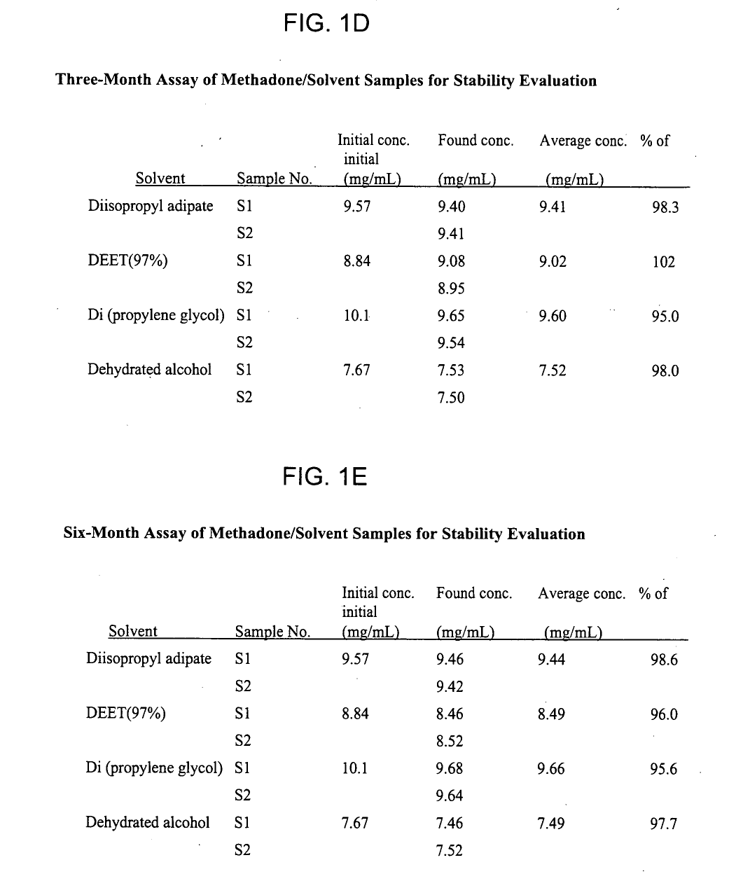

FIGS. 1A-1E show the results of the methadone/solvent stability studies over a period of time. Samples were stored at 40° C./75%RH. An HPLC assay was performed.

### Conclusions

The above results demonstrate the need for methadone to be in solution in order to pass through the skin. The solvent/carrier system used for this purpose must have the ability to solvate enough methadone for the desired effect, and at the same time, provide a stable milieu for the drug within the dosage form. The solubility data and the stability data demonstrate the suitability of certain solvents which may be used in the composition of a dosage form for methadone.

II. Concentrations of Methadone in Rat Plasma - Topical Administration versus Intravenous Injection

A. Study 1

i. Purpose

The purpose of the study was to evaluate the pharmacokinetics of methadone solution following intravenous and topical applications in a rat.

ii. Experimental Design

| | | | | | | | | Blood |
|---|---|---|---|---|---|---|---|---|
| | | | | | | | | Collection |
| | | | Number | Dose | | | | Time |
| | | | of | Route/ | Dose | | | Points in |
| Group | Sample | Species | Samples | Dose Site | Route | Test Article | Dosage | Minutes |
| A | A1 | Rat | 3 | I.V./tail | I.V. | Test Article 1: | 0.25 mL | 2/5/10/ |
| | A2 | | | vein | | 0.5 mg/mL * | | 30/60 |
| | A3 | | | | | Methadone- | | |
| | | | | | | Saline Solution | | |
| B | B1 | Rat | 3 | Topical/ | Topical | Test Article 2: | 1 × 1 cm | 10/20/45/ |
| | B2 | | | dorsal flank | | Near-Saturation | gauze pad | 60/120 |
| | B3 | | | | | Methadone- | with 0.2 mL | |
| | | | | | | Diisopropyl | solution | |
| | | | | | | Adipate Solution | | |
| C | C1 | Rat | 3 | Topical/ | Topical | Test Article 3: | 1 × 1 cm | 10/20/45/ |
| | C2 | | | dorsal flank | | Near-Saturation | gauze pad | 60/120 |
| | C3 | | | | | Methadone- | with 0.2 mL | |
| | | | | | | D.E.E.T. Solution | solution | |

Test Articles:
Test Article 1: Methadone from Teikoku Pharma USA, Inc.
Test Article 2: Methadone-Diisopropyl Adipate Solution (23.9%) from Teikoku Pharma USA, Inc.
Test Article 3: Methadone-DEET Solution (10%) from Teikoku Pharma USA, Inc.
Sample Preparation and Dosing Procedure:

For Group A, a 0.5 mg/mL solution of methadone was prepared by dissolving 1.3 mg of methadone powder in 2.6 mL of 0.9% Sodium Chloride Injection, USP. The pH of the solution was adjusted to 5 with 1N HCl and 1N NaOH to dissolve the methadone freebase.

The test article was administered intravenously (IV) through the tail vein to animals in Group A. The injection was performed slowly. A gauze pad (1×1 cm) Saturated with 0.2 mL of the appropriate test article was applied to the clipped site on the back of the animals in Groups 2 and 3. The gauze pad was secured by occlusive tape. The animal was wrapped in gauze, which was held in place with Zonas porous tape.

Blood Collection: Blood (0.5 mL) was collected into sodium heparin tubes at the appropriate time points. The samples were centrifuged at approximately 2800 rpm at 2-8 degrees Celsius for approximately 15 minutes. Plasma was collected.

iii. Results

| | | Concentrations of |
|---|---|---|
| | | Methadone in Rat |
| | | Plasma Samples |
| Sample | Time (min) | (ng/mL) |
| A1 | 2.0 | 69.5 |
| A1 | 5 | 56.0 |
| A1 | 10 | 42.3 |
| A1 | 30 | 35.8 |
| A1 | 60.0 | 22.7 |
| A2 | 2 | 73.5 |
| A2 | 5.0 | 68.7 |
| A2 | 10 | 61.8 |
| A2 | 30 | 35.3 |
| A2 | 60 | 22.8 |
| A3 | 2 | 67.8 |
| A3 | 5 | 68.9 |
| A3 | 10 | 57.6 |
| A3 | 30 | 30.9 |
| A3 | 60 | 24.5 |
| B1 | 10 | BQL |
| B1 | 20 | 4.14 |
| B1 | 45 | 11.5 |
| B1 | 60 | 11.3 |
| B1 | 120 | 44.1 |
| B2 | 10 | 3.02 |
| B2 | 20 | 3.25 |
| B2 | 45 | 10.4 |
| B2 | 60 | 15.1 |
| B2 | 120 | 57.8 |
| B3 | 10 | BQL |
| B3 | 20 | 3.18 |
| B3 | 45 | 7.24 |
| B3 | 60 | 12.4 |
| B3 | 120 | 26.2 |
| C1 | 10 | BQL |
| C1 | 20 | BQL |
| C1 | 45 | 3.83 |
| C1 | 60 | 3.88 |
| C1 | 120 | 6.85 |
| C2 | 10 | BQL |
| C2 | 20 | BQL |
| C2 | 45 | 2.66 |
| C2 | 60 | 3.27 |
| C2 | 120 | 6.22 |
| C3 | 10 | BQL |
| C3 | 20 | BQL |
| C3 | 45 | 2.84 |
| C3 | 60 | 4.16 |
| C3 | 120 | 5.97 |

*BQL = below quantification limit (1.00 ng/mL)

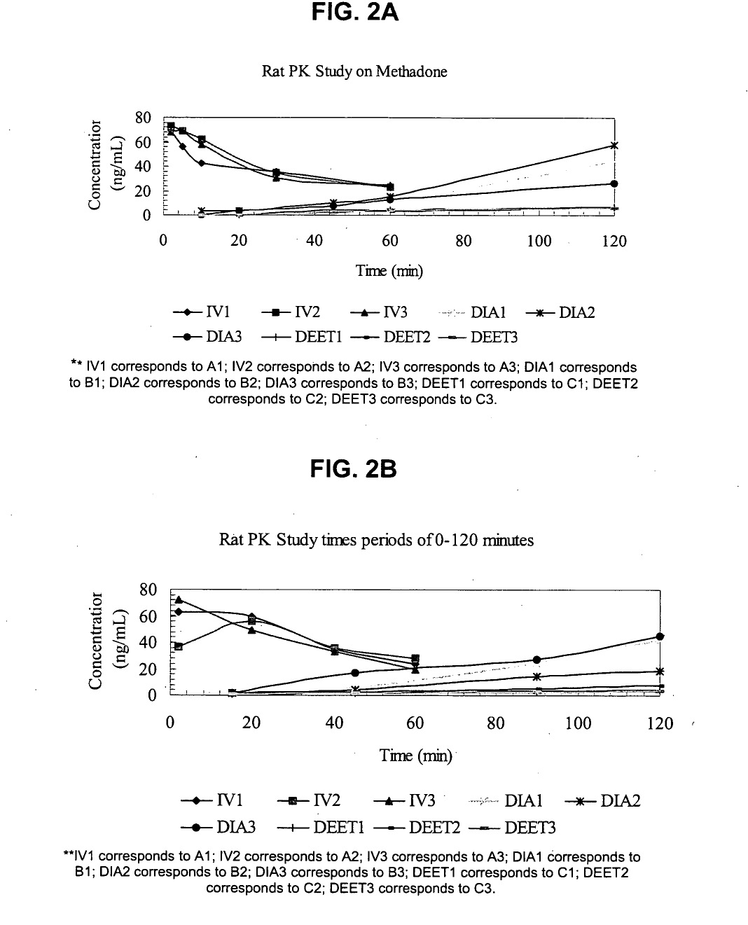

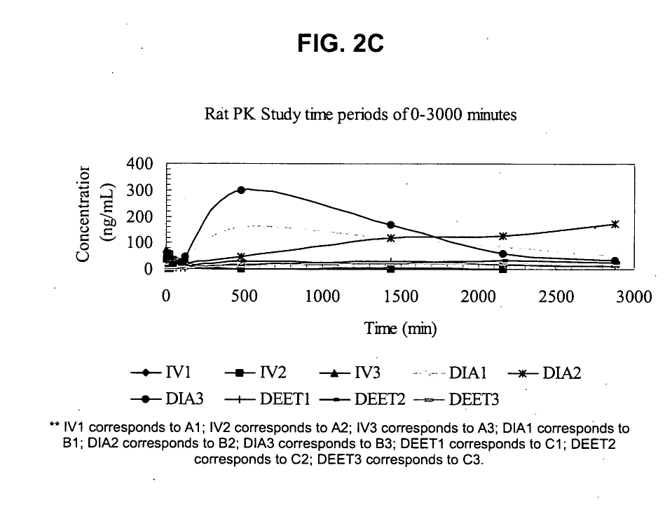

FIG. 2A provides a graphical representation of the above results.
iv. Conclusions

These data demonstrate that methadone is delivered through the skin of a rat at rates sufficient to provide systemic levels of methadone that are known to elicit analgesia, and at the same time, this topical application was non-irritating to the skin of the rat over the application time.

B. Study 2

i. Purpose

The purpose of the study was to evaluate the pharmacokinetics of methadone solution following intravenous and topical applications in a rat.

ii. Experimental Design

| | | | Number | Dose | | | Blood |
|---|---|---|---|---|---|---|---|
| | Sample | | of | Route/ | | | Collection |
| Group | Number | Species | Samples | Dose Site | Test Article | Dosage | Time Points |
| A | A1 | Rat | 3 | I.V./tail | Test Article 1: | 0.25 mL | 2 min/20 min/ |
| | A2 | | | vein | 0.5 mg/mL * | | 40 min/1 hr/ |
| | A3 | | | | Methadone- | | 2 hr/8 hr/ |
| | | | | | Saline Solution | | 24 hr/36 hr |
| B | B1 | Rat | 3 | Topical/ | Test Article 2: | 1 × 1 cm | 15 min/45 min/ |
| | B2 | | | dorsal flank | Near-Saturation | gauze pad | 90 min/2 hr/ |
| | B3 | | | | Methadone- | with 0.2 mL | 8 hr/24 hr/ |
| | | | | | Diisopropyl | solution | 36 hr/48 hr |
| | | | | | Adipate Solution | | |
| C | C1 | Rat | 3 | Topical/ | Test Article 3: | 1 × 1 cm | 15 min/45 min/ |
| | C2 | | | dorsal flank | Near-Saturation | gauze pad | 90 min/2 hr/ |
| | C3 | | | | Methadone- | with 0.2 mL | 8 hr/24 hr/ |
| | | | | | D.E.E.T. Solution | solution | 36 hr/48 hr |

Test Articles:
Test Article 1: Methadone from Teikoku Pharma USA, Inc .
Test Article 2: Methadone-Diisopropyl Adipate Solution (23.9%) from Teikoku Pharma USA, Inc.
Test Article 3: Methadone-DEET Solution (10%) from Teikoku Pharma USA, Inc.
Sample Preparation and Dosing Procedure: For Group A, a 0.5 mg/mL solution of methadone was prepared by dissolving 1.3 mg of methadone powder in 2.6 mL of 0.9% Sodium Chloride Injection, USP. The pH of the solution was adjusted to 5 with 1N HCl and 1N NaOH to dissolve the methadone freebase.

The test article was administered intravenously (IV) through the tail vein to animals in Group A. The injection was performed slowly. A gauze pad (1×1 cm) Saturated with 0.2 mL of the appropriate test article was applied to the clipped site on the back of the animals in Groups 2 and 3. The gauze pad was secured by occlusive tape. The animal was wrapped in gauze, which was held in place with Zonas porous tape.

Blood Collection: Blood (0.5 mL) was collected into sodium heparin tubes at the appropriate time points. The samples were centrifuged at approximately 2800 rpm at 2-8 degrees Celsius for approximately 15 minutes. Plasma was collected.

iii. Results

| | | Concentrations of |
|---|---|---|
| | | Methadone in Rat |
| | | Plasma Samples |
| Sample | Time (min) | (ng/mL) |
| A1 | 2 | 63.1 |
| A1 | 20 | 59.5 |
| A1 | 40 | 35.2 |
| A1 | 60 | 24.0 |
| A1 | 120.0 | 9.26 |
| A1 | 480.0 | 1.54 |
| A1 | 1440 | 2.31 |
| A1 | 2160.0 | 0.796 |
| A2 | 2 | 36.3 |
| A2 | 20 | 55.9 |
| A2 | 40 | 35.4 |
| A2 | 60 | 28.1 |
| A2 | 120.0 | 19.5 |
| A2 | 480.0 | 1.83 |
| A2 | 1440 | 0.923 |
| A2 | 2160.0 | 0.905 |
| A3 | 2 | 72.0 |
| A3 | 20 | 49.2 |
| A3 | 40 | 33.0 |
| A3 | 60 | 19.7 |
| A3 | 120.0 | 13.1 |
| A3 | 480.0 | 1.96 |
| A3 | 1440 | b.785 |
| A3 | 2160.0 | 0.944 |
| B1 | 15 | 2.75 |
| B1 | 45 | 5.66 |
| B1 | 90 | 24.8 |
| B1 | 120 | 41.4 |
| B1 | 480 | 159 |
| B1 | 1440 | 116 |
| B1 | 2160 | 82.7 |
| B1 | 2880 | 49.4 |
| B2 | 15 | 1.49 |
| B2 | 45 | 3.96 |
| B2 | 90 | 14.3 |
| B2 | 120 | 19.1 |
| B2 | 480 | 46.7 |
| B2 | 1440 | 117 |
| B2 | 2160 | 126 |
| B2 | 2880 | 173 |
| B3 | 15 | 1.70 |
| B3 | 45 | 17.0 |
| B3 | 90 | 27.1 |
| B3 | 120 | 44.9 |
| B3 | 480 | 300 |
| B3 | 1440 | 168 |
| B3 | 2160 | 59.4 |
| B3 | 2880 | 34.0 |
| C1 | 15 | 0.84 |
| C1 | 45 | 2.08 |
| C1 | 90 | 3.17 |
| C1 | 120 | 3.84 |
| C1 | 480 | 18.0 |
| C1 | 1440 | 27.8 |
| C1 | 2160 | 17.3 |
| C1 | 2880 | 11.1 |
| C2 | 15 | 1.72 |
| C2 | 45 | 2.42 |
| C2 | 90 | 4.7 |
| C2 | 120 | 7.73 |
| C2 | 480 | 29.6 |
| C2 | 1440 | 20.0 |
| C2 | 2160 | 35.1 |
| C2 | 2880 | 23.7 |
| C3 | 15 | 1.04 |
| C3 | 45 | 1.71 |
| C3 | 90 | 2.89 |
| C3 | 120 | 3.39 |
| C3 | 480 | 14.3 |
| C3 | 1440 | 20.6 |
| C3 | 2160 | 19.0 |
| C3 | 2880 | 12.6 |

The results are also provided in FIG. 2B and 2C.

iv. Conclusions

The above results demonstrate the reproducibility of the data from the previous experiment (Study 1 above) and confirm the lack of irritation and the delivery of a sufficient amount of methadone to provide an analgesic response.

III. Pharmacokinetic Study—Methadone Formulation in Human Plasma

Study A.

i. Purpose

The purpose of this study was to evaluate the pharmacokinetics of methadone solution following topical applications to a human.

ii. Experimental Design

Test Article: Methadone-DEET Solution (10.2%)

Protocol: 50 mg of methadone free base was dissolved in 450 mg of DEET to yield the sample solution. 500 mg sample solution was then soaked with a 7 cm×7 cm size KIMWIPE (Kimberley-Clark). Then the KIMWIPE soaked with the sample solution was then applied onto a human leg at the front area above knee. A layer of aluminum foil was placed on top of the KIMWIPE. A layer of adhesive was placed on top of the aluminum foil to hold the device in place on the leg of a 100 Kg male human.

| Plasma | |
|---|---|
| Sample Number | Blood Collection Time Point |
| H1 | 0 |
| H2 | 4 hours |
| H3 | 11 hours 50 minutes |
| H4 | 22 hours 15 minutes |

iii. Results

| | Concentrations of Methadone |
|---|---|
| Sample | in Human Plasma Samples (ng/mL) |
| H1 | Clotting of the sample was not sufficiently |
| | prevented and therefore no analysis was |
| | performed for collection at 0 hours |
| H2 | Below the quantification limit (1.00 ng/mL) |
| H3 | 3.50 ng/mL |
| H4 | 9.86 ng/mL |

iv. Conclusions

The results of this experiment confirm that methadone is substantially absorbed through intact skin from a 10% solution in DEET. Furthermore, it demonstrates the lack of irritation to a human after a one-day application of the topical methadone composition to the skin.

Study B.

Concentrations of Methadone in Human Plasma Samples

| Sample | Concentration | Blood Collection |
|---|---|---|
| Information | (ng/mL) | Time Point (hr) |
| 1 | 5.36 | 12 |
| 2 | 4.95 | 18 |
| 3 | 2.24 | 139.5 |

| Formu- | | |
|---|---|---|
| lation | | Weight |
| Used | Contents | % |
| | Sytrene-Isoprene- | 15 |
| | Sytrene Copolymer | |
| | Hydrogenated Rosin | 10 |
| | Glycerol Ester, KE311 | |
| | Alicyclic Saturated | 30 |
| | Hydrocarbon Resin, P100 | |
| | Liquid paraffin | 30 |
| | Methadone | 10 |
| | DEBT | 5 |

| | |
|---|---|
| Protocol | 25 mg Methadone free base in 5 × 5 cm patch. |
| | Application time was 12 hours. |
| | Patch was applied on a human left leg inner side above knee. |
| | Subject was a 55 kg Asian male. |

IV. Additional Stability Data

A. Protocol-

Three samples of a methadone topical patch of formulation F6 with a 2×2 cm surface area were analyzed using the HPLC method. Analysis of each sample was repeated once to ensure accuracy.

F6 Formulation

| Contents | F6 Weight % |
|---|---|
| SIS | 20 |
| KE311 | 25 |
| P100 | 15 |
| Liquid paraffin | 25 |
| Methadone | 10 |
| DEET | 5 |
| Total | 100 |

HPLC conditions:
Column: Dionex Acclaim 120, C18, 3um Analytical
Mobile Phase: 40% Acetonitrile, 60% Potassium Phosphate Buffer with adjusted pH=3.1
Flowrate: 1ml/min
Column Oven: 40C
Detector: 210 nm

C. Results

3 months stability at room temperature of the patch formulation has an average recovery of 96.2% assuming 100% recovery for initial samples. The results of the 6 months stability at room temperature of formula show similar value in recovery. The average recovery for the 6 month period was found to be 96.4%.

| | Formula | | Recovery |
|---|---|---|---|
| Sample | # | Condition | % |
| 1 | F6 | #1/Room Temperature/3 months | 94.3 |
| 1 repeat | F6 | #1/Room Temperature/3 months | 93.7 |
| 2 | F6 | #2/Room Temperature/3 months | 96.6 |
| 2 repeat | F6 | #2/Room Temperature/3 months | 97.0 |
| 3 | F6 | #3/Room Temperature/3 months | 97.5 |
| 3 repeat | F6 | #3/Room Temperature/3 months | 98.0 |
| 4 | F6 | #1/Room Temperature/6 months | 97.0 |
| 4 repeat | F6 | #1/Room Temperature/6 months | 96.3 |
| 5 | F6 | #2/Room Temperature/6 months | 95.2 |
| 5 repeat | F6 | #2/Room Temperature/6 months | 95.6 |
| 6 | F6 | #3/Room Temperature/6 months | 96.8 |
| 6 repeat | F6 | #3/Room Temperature/6 months | 97.3 |

It is evident from the above results and discussion that the subject methods provide an improved method of treating pain, which pain may be treated through direct action on μ-receptors at the site of pain. The above-described invention provides a number of advantages including ease of use and effective and efficient manners of topically delivering methadone to a subject to treat the subject for pain. The subject invention provides rapid penetration of an effective amount of methadone through the skin surface, thereby providing for rapid pain relief. The subject methods also provide a low dose alternative to systemic methadone administration. As such, the subject invention represents a significant contribution to the art.

All publications and patents cited in this specification are herein incorporated by reference as if each individual publication or patent were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. A method for administering a methadone agent to a subject, said method comprising:
topically applying to a skin surface of said subject a formulation comprising a methadone agent as the only active agent present in said formulation to administer said methadone agent to said subject.

2. The method of claim 1, wherein said formulation comprises a thermoplastic elastomer matrix.

3. The method of claim 2, wherein said matrix is a styrene-butadiene- styrene block copolymer matrix or a styrene-isoprene-styrene block copolymer matrix.

4. The method of claim 2, wherein said matrix is self-adhesive.

5. The method of claim 2, wherein said matrix is present on a backing layer.

6. The method of claim 5, wherein said backing layer is substantially impermeable to said compound.

7. The method of claim 1, wherein said formulation comprises from about 0.1 % to about 30.0 % (w/w) of said methadone agent.

8. The method according to claim 1, wherein said method is a method of treating said subject for pain.

9. The method of claim 1, wherein said topical formulation is a cream, gel, ointment or lotion.

10. A method of administering a methadone agent to a subject, said method comprising:
(a) contacting a skin surface of said subject with a thermoplastic elastomeric matrix comprising said methadone agent; and
(b) maintaining said matrix on said skin surface for a period of time sufficient for said methadone agent to be delivered to said subject.

11. The method of claim 10, wherein said matrix is a styrene-butadiene- styrene block copolymer matrix or a styrene-isoprene-styrene block copolymer matrix.

12. The method of claim 10, wherein said matrix is self-adhesive.

13. The method of claim 12, wherein said matrix is present on a backing layer.

14. The method of claim 13, wherein said backing layer is substantially impermeable to said pharmacological agent.

15. The method of claim 10, wherein said methadone agent is present in said matrix is an amount ranging from about 0.1 % to about 30.0 % (w/w).

16. The method of claim 10, wherein said method is a method of treating said subject for pain.

17. A thermoplastic elastomer matrix comprising a methadone agent .

18. The matrix of claim 17, wherein said matrix is a styrene-butadiene-styrene block copolymer matrix or a styrene-isoprene-styrene block copolymer matrix.

19. The matrix of claim 17, wherein said matrix is self-adhesive.

20. The matrix of claim 17, wherein said matrix is present on a backing layer.

21. The matrix claim 20, wherein said backing layer is substantially impermeable to said methadone.

22. The matrix of claim 17, wherein said methadone is present in said matrix is an amount ranging from about 0.1 % to about 30.0 % (w/w).

23. A kit comprising:
(a) a topical methadone formulation comprising a methadone agent, wherein said methadone is the only active agent present in said formulation; and
(b) instructions for practicing the method according to claim 1.
